(19) United States
(12) Patent Application Publication
(10) Pub. No.: US 2006/0128606 A1
(43) Pub. Date: Jun. 15, 2006

(54) Fibrils

(76) Inventor: Christopher M. Dobson, Oxford (GB)

Correspondence Address: QUARLES & BRADY LLP, 411 E. WISCONSIN AVENUE, SUITE 2040, MILWAUKEE, WI, 53202-4497, US

(21) Appl. No.: 11/336,530

(22) Filed: Jan. 20, 2006

Related U.S. Application Data
(63) Continuation of application No. 09/787,560, filed on Jun. 4, 2001.
(60) Provisional application No. 60/126,871, filed on Mar. 30, 1999.

(30) Foreign Application Priority Data
Sep. 21, 1998 (GB) 9820555.2
Apr. 29, 1999 (GB) 9909927.7

(51) Int. Cl. A61K 38/17; C07K 14/47

(52) U.S. Cl. 514/6; 530/350; 977/915

(57) ABSTRACT

An amyloid fibril substantially free of other protein is disclosed. Also disclosed are processes for preparing the fibril, and methods and uses of the fibril particularly in connection with treating diabetes, blood clotting disorders, cancer and/or heart disease.

### CROSS REFERENCE TO RELATED APPLICATIONS

This is a continuation of U.S. Ser. No. 09/787,560 filed Jun. 4, 2001, which was a national phase application based on PCT/GB99/033133 filed Sept. 21, 1999, which claimed priority based on U.S. provisional application 60/126,871 filed Mar. 30, 1999, U.K. application 9820555.2 filed Sept. 21, 1998, and U.K. application 9909927.7 filed Apr. 29, 1999.

### STATEMENT REGARDING FEDERALLY SPONSORED RESEARCH

Not applicable.

### BACKGROUND OF INVENTION

The present invention relates to amyloid fibrils, processes for their preparation and their use. The invention in particular relates to both naturally occurring amyloid fibrils and non-naturally occurring amyloid fibrils comprising a protein, their preparation and their use, for example, as a plastic, a slow-release form of pharmaceutically active proteins or a material for fabrication, or in the delivery of pharmaceutically active compounds, electronics or catalysis.

By “protein”, as used herein, is meant one or more proteins, protein fragments, polypeptides or peptides. The protein is any protein capable of forming fibrils and may be a pharmaceutically active protein.

The amyloidoses are a group of protein misfolding disorders characterised by the accumulation of insoluble fibrillar protein material in intra- or extra-cellular spaces. The deposition of normally soluble proteins or their precursors in this insoluble form is believed to lead to tissue malfunction and cell death. A number of different proteins and peptides have been identified in amyloid deposits to date. These include the Aβ peptide in Alzheimer's disease, the prion protein in the transmissible spongiform encephalopathies, the islet-associated polypeptide in type II diabetes, and other variant, truncated, or misprocessed proteins in the systemic amyloidoses (S. Y. Tan and M. B. Pepys (1994) Histopathology 25, 403-414 and J. W. Kelly (1996) Curr. Op. Struct. Biol 6, 11-17).

Proteins known to form amyloid fibrils in vivo appear to have no obvious sequence or structural similarities, and where the soluble folds of the amyloidogenic precursors are known they span the range of secondary, tertiary, and quaternary structural elements. In spite of this diversity, there is a body of evidence that indicates that all amyloid fibrils are long, straight and unbranching, with a diameter of from 7 to 12 nm, and they all exhibit a cross-β diffraction pattern. The protein molecules constitute individual or multiple beta-strands oriented perpendicular to the long axis of the fibril and forming long beta-sheets that propagate in the direction of the fibril twisting around each other.

The mechanism by which amyloidogenic proteins undergo the conversion from a soluble globular form to the cross-β conformation displayed by the disease-associated fibrils has not yet been elucidated in detail. Nevertheless, the conformational reorganization associated with amyloid formation is well documented (J. W. Kelly (1997) Structure 5, 595-600). Studies of some of the amyloidogenic variants of transthyretin, lysozyme and the Ig light chain have investigated the process of conformational change that leads to amyloid deposition. Amyloid formation, at least for the latter three proteins, appears to start from partially structured forms of the proteins.

### BRIEF SUMMARY OF THE INVENTION

The present invention concerns naturally occurring amyloid fibrils, which to date have been associated with disease, and non-naturally occurring amyloid fibrils comprising a protein which may have a variety of useful applications. The fibrils may be used, for example, as a plastic or as a slow-release form of pharmaceutically active proteins, or in the delivery of pharmaceutically active compounds, electronics or catalysis.

In a first aspect, the present invention provides amyloid fibrils substantially free of other protein.

In one embodiment the fibril is an amyloid fibril substantially free of other protein other than an amyloid fibril formed from an SH3 domain of a p85α subunit of bovine phosphatidylinositol 3-kinase at pH 2.0.

In a further embodiment the fibril is an amyloid fibril substantially free of other protein other than an amyloid fibril formed from an SH3 domain of a p85α subunit of bovine phosphatidylinositol 3-kinase.

The amyloid fibril may be naturally or non-naturally occurring. The naturally occurring amyloid fibrils of the present invention include, for example a fibril of the Aβ peptide associated with Alzheimer's disease, the prion protein associated with the transmissible spongiform encephalopathies, the islet-associated polypeptide associated with type II diabetes, transthyretin and fragments thereof associated with senile systemic amyloidosis, transthyretin variants and fragments thereof associated with familial amyloidotic polyneuropathy or other variant or truncated or misprocessed proteins associated with systemic amyloidoses.

In a second aspect the present invention provides a non-naturally occurring amyloid fibril comprising a protein.

In one embodiment the fibril is a non-naturally occurring amyloid fibril, comprising a protein other than an amyloid fibril formed from an SH3 domain of a p85α subunit of bovine phosphatidylinositol 3-kinase at pH 2.0.

In another embodiment the fibril is a non-naturally occurring amyloid fibril comprising a protein other than an amyloid fibril formed from an SH3 domain of a p85α subunit of bovine phosphatidylinositol 3-kinase.

In another embodiment the fibril is a non-naturally occurring amyloid fibril comprising an SH3 domain of a p85α subunit of bovine phosphatidylinositol 3-kinase and at least one protein selected from a derivative or amino acid variant of an SH3 domain of a p85α subunit of bovine phosphatidylinositol 3-kinase, human muscle acylphosphatase or a derivative or amino acid variant thereof, bovine insulin or a derivative or amino acid variant thereof, a protein corresponding to the first two (CspB-1), the first three (CspB-2) or the last two (CspB-3) β-strands of CspB (the major cold shock protein of Bacillus subtilis) or a derivative or amino acid variant thereof and the activation domain of wild type human carboxypeptidase A2 (WT ADA2h) or a derivative or amino acid variant thereof.

In a further embodiment the fibril is a non-naturally occurring fibril comprising a derivative or amino acid variant of an SH3 domain of a P85α subunit of bovine phosphatidylinositol 3-kinase, human muscle acylphosphatase or a derivative or amino acid variant thereof, bovine insulin or a derivative or amino acid variant thereof, a protein corresponding to the first two (CspB-1), the first three (CspB-2) or the last two (CspB-3) β-strands of CspB (the major cold shock protein of Bacillus subtilis) or a derivative or amino acid variant thereof or the activation domain of wild type human carboxypeptidase A2 (WT-ADA2h) or a derivative or amino acid variant thereof.

The fibrils of the present invention may comprise non-naturally occurring proteins. The proteins may be, for example, proteins which have been chemically modified such as proteins which have been glycosylated or proteins which comprise a modified amino acid residue, a pharmaceutically active compound, a metal or a functional group such as a thiol group which is capable of binding one or more reactants. The protein is, for example a derivative or amino acid variant of an SH3 domain (PI3-SH3) of a p85α subunit of bovine phosphatidylinositol 3-kinase, human muscle acylphosphatase, bovine insulin, a protein corresponding to the first two (CspB-1), the first three (CspB-2) or the last two (CspB-3) β-strands of CspB (the major cold shock protein of Bacillus subtilis) or the activation domain of wild type human carboxypeptidase A2 (WT-ADA2h).

The fibrils of the present invention are typically long, straight and unbranching. The diameter of the fibrils is generally from 1 to 20 nm, preferably from 5 to 15 nm and more preferably from 7 to 12 nm. The diameter of the fibrils may be varied by selecting suitable proteins.

It is believed that some of the fibrils of the present invention may comprise a hollow core which may be useful in a variety of applications.

It has been found that the fibrils of the present invention may be obtained by preparing a solution comprising a protein, typically one or more single chain polypeptides, said solution being in a state so that nucleation of the protein and fibril growth will occur over an acceptable time, and allowing nucleation and fibril growth to take place.

By“nucleation”, as herein used, is meant the initiation of processes that lead to fibril formation. Fibril formation from a solution involves, successively, protein self-association, formation of aggregates and fibril growth. Thus, desirably, the initiation solution is on the verge of instability. Nucleation and growth are slow processes and conditions are normally chosen so that fibril formation occurs over a period of hours or days. It will be appreciated that if nucleation occurs too rapidly then this will often have an adverse affect on fibril formation.

Nucleation can be caused by a variety of means including variations in solvents, concentration, salt ligands, temperature and pH, as discussed below. It may for example, be caused by the addition of urea, preferably at concentrations of from 4 to 7 M. Shaking, agitation and exposure to certain surfaces, for example the surface of a glass or plastic vessel, may cause local denaturation and thereby initiate fibril formation.

The solution comprising a protein may comprise any solvent or mixture of solvents in which nucleation can occur. For example, the solution may comprise DMSO, dioxan and/or water. Preferably the solution is an aqueous solution.

One or more organic solvents which can promote nucleation-and fibril growth may be incorporated into the solution. In the case of naturally occurring proteins conditions are typically chosen to denature at least partially the protein whilst retaining conditions in which self-association can occur. The organic solvent is generally water-miscible and is preferably an alcohol or an aliphatic nitrile such as acetonitrile. The alcohol is typically a C₁₋₆ alkanol which may be substituted or unsubstituted for example by one or more halogen atoms, especially fluorine atoms.

Examples include methanol, ethanol, propanol or butanol, or fluorinated alcohols such as trifluoroethanol or hexafluoroisopropanol. Preferably the alcohol is trifluoroethanol. The concentration of alcohol is typically from 5 to 40% v/v and preferably about 25% v/v. The concentration of aliphatic nitrile can vary between wide limits and is typically from 5 to 95% v/v.

The concentration of protein in the solution is not limited in any way but it must be such that nucleation can occur. Generally the concentration is from 0.1 mM to 10 mM. Preferably the concentration of protein is about 1 mM.

The temperature of the solution is generally from 0° C. to 100° C. Preferably the temperature is from 0° C. to 70° C., more preferably from 0° C. to 40° C. and most preferably from 5° C. to 30° C.

The pH of the solution is any pH suitable for nucleation. Preferably the solution is acidic and more preferably the pH of the solution is from 0.5 to 6.5.

The solution may be seeded with, for example, previously formed particles of protein; this can greatly speed up the process.

The fibrils of the present invention are suitably isolated by centrifugation, filtration or evaporation of solvent. The fibrils thus obtained may then be washed and dried.

The fibrils of the present invention may be formed from pharmaceutically active proteins such as insulin, calcitonin, angiostatin or fibrinogen. The fibrils may therefore be used as a slow release form of such proteins due to the low solubility of the fibrils in vivo.

Alternatively, the fibrils of the present invention may be used in the delivery of pharmaceutically active compounds. They may, for example, comprise a protein which has been chemically modified to incorporate a pharmaceutically active compound or a pharmaceutically active compound may, for example, be retained inside a fibril with a hollow core by hydrogen bonding. Pharmaceutically active compounds which may be delivered using the fibrils of the present invention include, for example, cancer drugs such as cis Pt, anti-biotics, anti-inflammatories and analgesics.

The fibrils of the present invention may comprise one or more functional groups capable of binding one or more reactants. The functional groups may occur naturally in the protein of the fibrils or be incorporated by chemical modification. Reactants may be brought together inside fibrils with a hollow core or on the outside of fibrils.

The fibrils of the present invention may be used in the treatment of, for example, diabetes, blood clotting disorders, cancer and heart disease.

The fibrils of the present invention may comprise a metal, such as copper, silver or gold, and form wires which may be useful in electronics.

The fibrils of the present application may also be used as plastics or made into structures.

### DESCRIPTION OF THE DRAWINGS

The present invention is further illustrated, merely by way of example, with reference to the Figures in which:

FIG. 1(a) to 1(d) show negative stain electron microscopy images of SH3 amyloids, showing a range of morphologies similar to those observed with disease related fibrils. FIG. 1(e) shows a cryo EM image and (f) shows the diffraction pattern of the form seen in (d) with an obvious helical twist, which was used for 3D reconstruction. The layer line spacing is around 60 nm, the asymmetric unit of the double helix. The various ribbons and smooth fibrils were formed at pH 2 (a,b) and pH 2.66 (c). The helical fibres formed at pH 2 are seen by negative stain in (d) and cryo EM in (e).

FIG. 2 shows class averages (a,e), reprojections of 3D reconstructions (b,f), 1D projections (c,g) and diffraction patterns of the reprojections (d,h) for the 58 and 61 nm long repeats, respectively. (In this figure only, the fibre axis is horizontal). A region in (a) showing a ˜3 nm periodicity is enlarged and marked with lines. The good agreement between the input class averages and the reprojections of the 3D maps (compare a to b and e to f), and also between the diffraction pattern of a single fibril and of reprojected maps (g,h), supports the validity of the reconstruction procedure. The line projection comparisons (c,g) show that the 3D maps fit the input images better when the 2.7 nm subunit repeat is used in the reconstruction procedure than if the fibre is treated as continuous helix.

FIG. 3 shows 3D reconstructions and contoured density sections of the 61 nm (a,c) and the 58 nm form (b,d). The fibrils are shown as rendered surfaces in a and c, and as contoured density cross-sections in c and d. The two independent reconstructions are very similar and both show four protofilaments winding around a hollow core, with protruding edge regions. The 2.7 nm subunit repeat is most pronounced on the edge structure.

FIG. 4 shows modelling the polypeptide fold in the fibrils. FIG. 4(a) shows a cross-section of the fibre and FIG. 4(b) shows a side view of a single protofilament. β-sheets derived from the PI3-kinase SH3 structure have been fitted into the map, after opening the β sandwich fold and reorientating and strengthening the strands. The remaining regions of polypeptide sequence are shown as disconnected dots, to indicate the number of residues present but not the conformation. At the angle of view in (a), the upper right and lower left profilaments curve inwards below the plane of view, making the quality of the fit less apparent. The side view in (b) shows that the Psheets fit well into the density.

FIG. 5A shows a far-UV circular dichroism spectra of muscle acylphosphatase acquired during a fibrillogenesis process. The first and last spectra reported in the figure were acquired after 3 and 600 minutes from the initiation of the reaction, respectively. The spectra show a slow two-state transition between two conformations containing significant amounts of α-helical and β-sheet structure, respectively. After 600 minutes the spectra did not change their shapes but underwent a progressive reduction of signal and a shift of the negative peak towards the higher wavelengths, as a result of the accumulation of protein aggregates of major size. FIG. 5B shows an amide I region of the infra-red spectrum of muscle acylphosphatase. The two peaks at 1613 and 1685 cm⁻¹ indicate a cross-β structure.

FIGS. 6A-C are electron micrographs showing the morphological development of the muscle acylphosphatase aggregate. FIG. 6A shows an aggregate of granular aspect after 72 minutes from initiation of the reaction. FIG. 6B shows short fibrils after 32 hours. FIG. 6C shows amyloid fibrils after two weeks. The scale bar represents a distance of 100 nm. FIG. 6D shows an optical microscope photograph of a sample containing muscle acylphosphatase-derived aggregate obtained after two weeks of incubation. The arrows indicate the blots of green birefringence coming from regions of amyloid fibril.

FIG. 7 shows the sequence and secondary structure content of the cold shock protein CspB from Bacillus subtilis. The numbers indicate the first and last amino acids of the three peptides: CspB-1 (1-22), CspB-2 (1-35), and CspB-3 (3667).

FIG. 8 shows the characterization of dilute solutions of the CspB peptides by CD spectroscopy. Acetonitrile concentration was varied as indicated. A-C: CD spectra recorded at acetonitrile concentrations ranging from 2.5 to 97.5% of solutions containing 0.4 mg/mL of (A) CspB-1, (B) CspB-2, and (C) CspB-3. D: Ellipticity at 215 nm plotted against the acetonitrile concentration. Circles: CspB-1, squares: CspB-2, triangles: CspB-3.

FIG. 9 shows the difference of the residue specific ³J_{NHα}coupling constants extracted from the antiphase splitting in a COSY spectra by fitting to simulated cross-sections for (A) CspB-1 and (B) CspB-3 from those predicted from the random coil model. A positive difference from these random coil values indicates an increase in the population of the β-region of φΨ space, and negative differences an increase in the population of the α-region.

FIG. 10 shows the evidence obtained for amyloid fibrils formed by CspB-1 upon reduction of the acetonitrile concentration. A: Example of the VIS spectra of the congo red assay. The dashed line represents the spectrum before, the solid line the one after, addition of a sample of CspB-1 in 109 acetonitrile. The shift towards higher wavelengths and greater intensity indicates fibril formation. B: Electron micrograph of negatively stained fibrils. The scale bar corresponds to a length of 200 nm. C: X-ray fiber diffraction pattern obtained from a sample dried down from a 5 mg/mL solution in 50% acetonitrile. D: Cross section of the diffraction pattern in C with assignment of the peaks corresponding to the distances typical for β-sheet structure.

FIG. 11 shows the electron microscopy analysis of WT-ADA2h preparations. WT-ADA2h fibrils prepared by incubation of protein samples at 90° C. for a: 1 h and b to d: 48 h. Longer and straighter fibrils can be observed in the later preparations. Thin arrows point to possible crossover sites, whereas solid arrows indicate helical ribbon-like conformations.

### DETAILED DESCRIPTION

The Examples which follow further illustrate the present invention with reference to the Figures.

### EXAMPLES

### Example 1

Microscopy and Image Classification

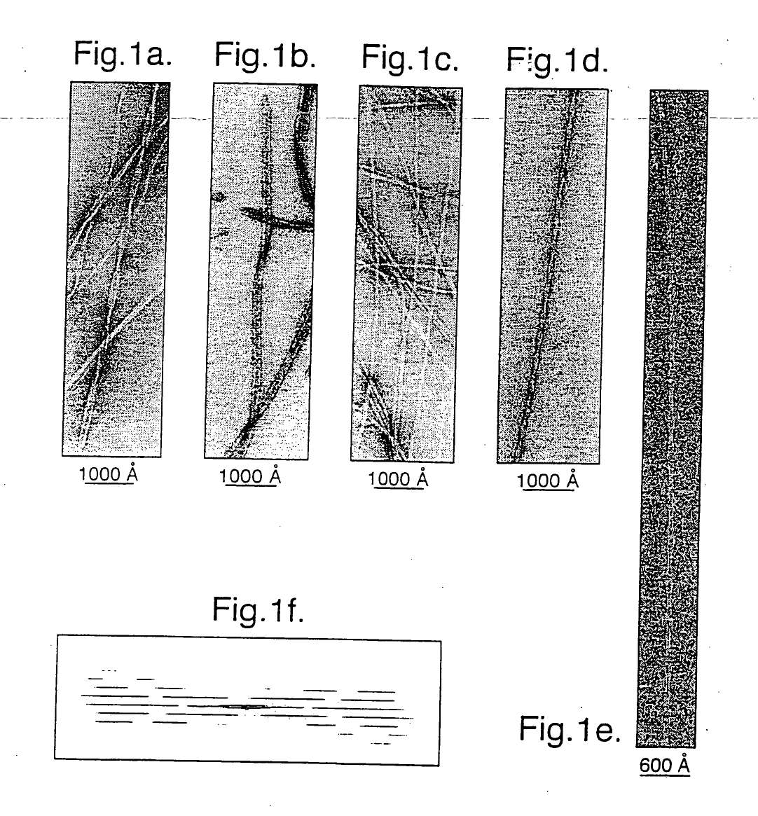

Samples of twisted fibrils of the PI3-kinase SH3 domain formed after several months incubation at pH 2 (J. I. Guijarro et al (1998) Proc. Natl. Acad. Sci. USA. 95, 4224-4228) were vitrified on holey carbon grids, and low electron dose images were recorded at 120 kV and 1.3-1.5 μm underfocus on a JOEL 1200 EX microscope with an Oxford Instruments cryotransfer stage at 30,000×. Films were digitised on a Leafscan 45 linear CCD scanner (Ilford Ltd, Cheshire, UK) at a spacing of 10 μm, and interpolated to 0.67 nm/pixel for processing. Calculated diffraction patterns (FIG. 1f) were obtained by straightening fibres with Phoelix software, but the axial resolution was severely limited in the pitch, which ranged from 54.5 to 66 nm. In order to avoid resolution loss due to non-linear interpolation, digitised fibres were cut into individual repeats and treated as single particles. 890-cut-out repeats were iteratively aligned and sorted into classes by multivariate statistical analysis, using either Imagic or Spider. This allowed identification of classes of repeats that were naturally straight and had the same length.

3D Reconstruction

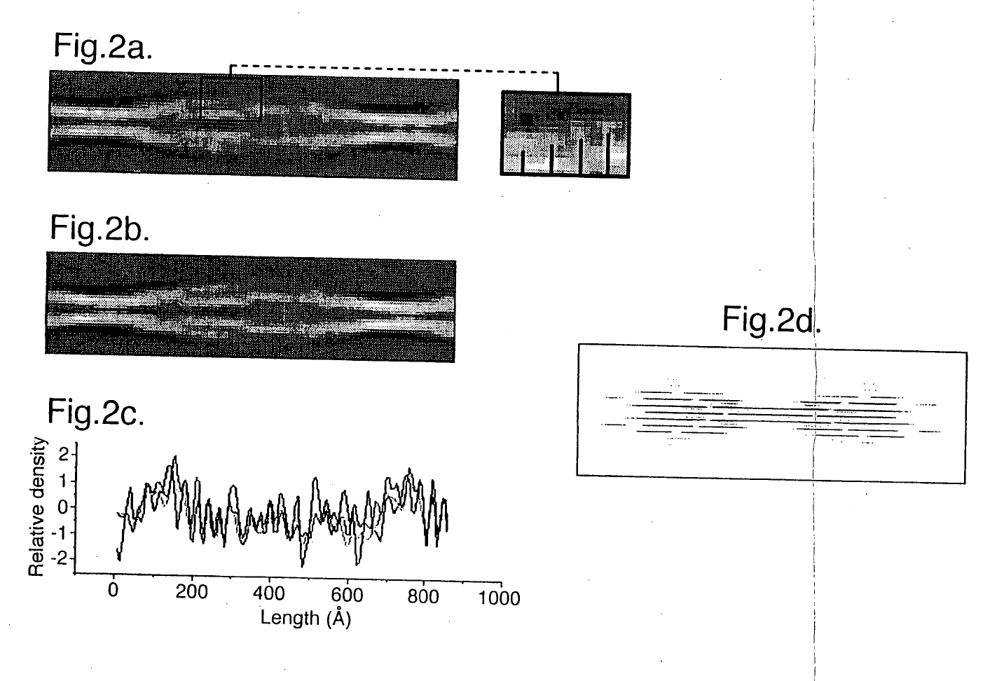

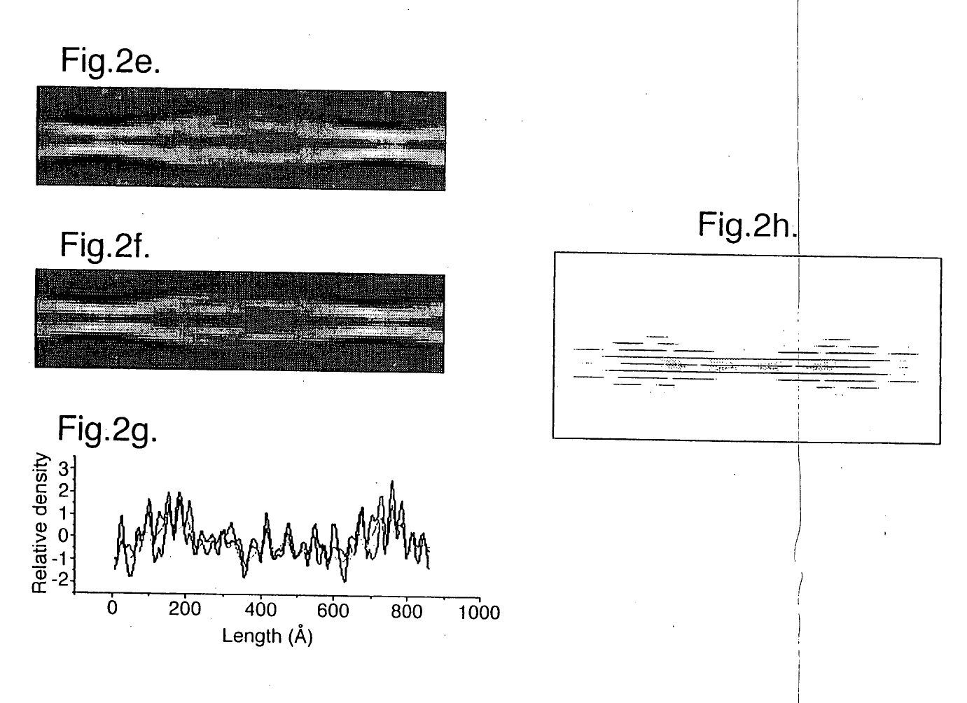

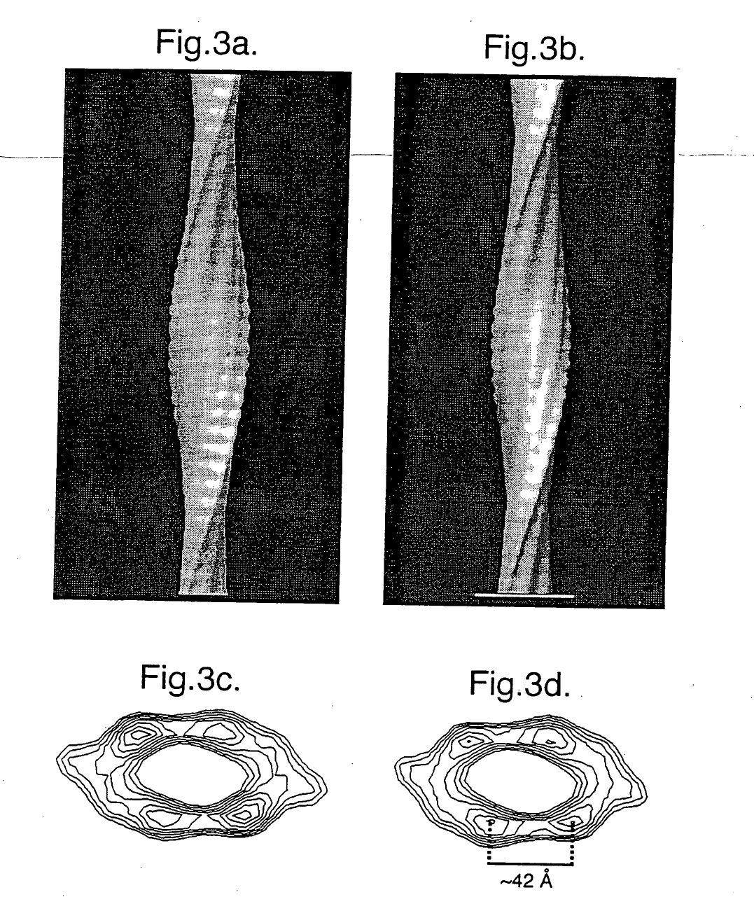

Two class averages of with low inter-image variance, containing 92 and 77 images (˜20% of the data set), corresponding to a 58 and 61 nm repeat respectively, were selected for 3D reconstruction. The repeat length was determined by cross-correlation of the class averages with the excised cross-over region. The subunit repeat was clearly observable in axial 1D projections of the class averages after square root amplitude filtering (FIG. 2c,g). The repeat was determined as approximately 2.7 nm in both cases, and the value used was chosen to give an integral number of subunits in the 58 and 61 nm repeats (21 and 22 subunits respectively). 3D reconstructions were calculated by back projection, assuming either a continuous helix or the 27 nm subunit repeat. The overall features of protofilament packing and density cross section were unaffected by imposition of a subunit repeat, but the line projections (FIG. 2c,g) and diffraction patterns (FIG. 2d,h) of the reprojected images gave a better match to the input data when the 27 nm repeat was imposed. The diffraction pattern of the reprojected helix gave excellent agreement with the original one from the straightened fibre, and showed strong intensity to 22 nm resolution in the equatorial (radial) direction (FIG. 1f). Resolution tests by Fourier shell correlation and phase residual between cross sections of the two maps (FIG. 3c,d) show agreement to 2.5 nm, but there is reliable information to 2.2 nm in the equatorial direction for each map. The absolute handedness is not determined by this method and is arbitrary. Other procedures have been used for correlation of the helical disorder based on cross-correlation and back projection. The 3D maps were examined with AVS (Advanced Visualisation System) and β-sheet fitting was done in O.

The native fold of the 84 residue SH3 domain of the p85α subunit of bovine PI3 kinase contains five β-strands arranged in a β-sandwich. At low pH, the protein partially unfolds and assembles into amyloid fibrils. The images in FIG. 1a to 1d show a range of twisted and flat ribbons, and smooth and twisted tubular fibres. For structural analysis, a form with a pronounced helical twist was selected. Diffraction patterns (FIG. 1f) calculated from cryo EM images (FIG. 1e) contain layers at spacings between 54.5 to 66 nm, the distance between helical cross-overs in the double-helical structure, ie. the length of the helical repeat.

The diffraction data show structure information to 2.2 nm resolution in the equatorial direction (perpendicular to the fibre axis), but the meridional pattern fades out around 15 nm due to variations in the helical pitch (angular disorder). To retrieve the structural information lost due to angular disorder, the digitised images of the fibrils were divided up into individual helical repeats. These repeats were aligned and sorted into classes according to their length. The class averages of a 28 and a 61 nm repeat are shown in FIG. 2 a,e, along with reprojections of 3D maps calculated from these two repeats (2b,f), and their diffraction patterns (2d,h). A subunit repeat is visible in the class average (FIG. 2a, expanded) and sometimes in the raw images (not shown). A subunit periodicity of 2.7±0.3 nm projections of the class averages was determined(FIG. 2c,g).

The two independent 3D maps, derived from the 58 and 61 nm repeats, reveal the same features (FIG. 3). The surface views and cross sections show two pairs of thin profilaments winding around a hollow core. Regions of weaker density form the extended edges that give the fibrils their characteristic twisting appearance. The profilaments are about 4 nm part and 2 nm thick (FIG. 3c,d), too thin to accommodate the native SH3 structure, whose minimum dimension is 3 nm. X-ray fibre diffraction of SH3 amyloid indicates an ordered core of cross-β structure with a 0.47 nm meridional and a 0.94 nm equatorial repeat defining the inter-strand and inter-sheet distances respectively. The 2 nm width can only fit two β-sheets, which must be orientated differently from those in the native fold to make all the strands perpendicular to the fibre axis. The twist between β-strands is also very restricted by the narrow dimension and ling pitch of the profilaments, giving flat sheets with an inter-strand angle of less that 2°.

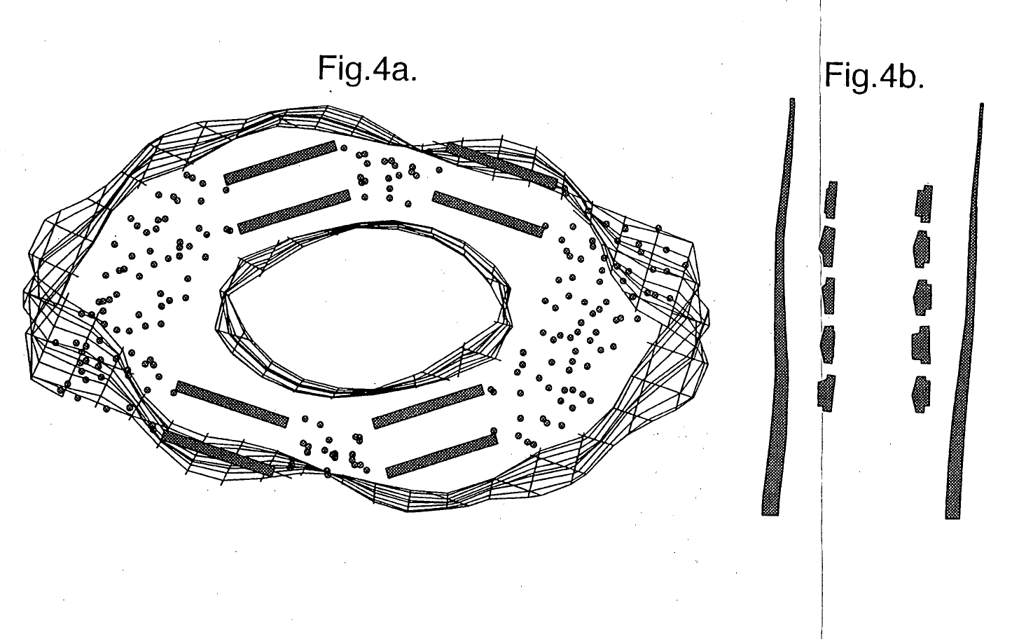

A model in which the SH3 are reorganised to fit into the EM density is shown in FIG. 4. The remaining short and long loops are the right size range and provide the contracts between adjacent profilaments and to give rise to diffuse density in the protruding edges of the structure. Consistent with the observation that fibres are seen to split into sub-fibrils, that individual polypeptide chains could contribute β-strands to each member of a pair of protofilaments. Since the axial repeat corresponds to 5 β-strands, it is possible that this is related to the 2-and 3-stranded sheets of the native fold by a rearrangement similar to a domain swapping mechanism. Non-covalent interactions would then provide the bonds assembling the adjacent sub-fibrils into the double helical structure.

The structure determined here, in which the protofilaments are effectively continuous β-sheets, may provide a basic model for all amyloid fibres, irrespective of the chain length and native conformation of the component protein. Indeed, negative stain EM, atomic force microscopy and fibre diffraction of Aβ(1-40) fibrils suggest a very similar morphology with two sub-fibrils and 3-5 protofilaments. EM studies of ex vivo transthyretin fibrils indicate that these consist of four protofilaments of diameter 5-6 nm. The transthyretin protofilament core has been modelled, based on X-ray fibre diffraction data, as four β-sheets with a 15° twist between adjacent strands. The two-sheet protofilament model presented here could however be extended to a larger number of sheets for thicker protofilaments. At present there is no evidence to discriminate between twisted and flat β-sheets in the larger protofilament type, but the maps are not consistent with as twisted sheet configuration for the SH3 protofilaments since they are only 2 nm thick and have a very small overall twist. Although flat, untwisted β-sheets are unusual in the protein structure database, part of the β-helix of alkaline protease has such a structure.

The cryo-EM work provides 3D information on how a polypeptide chain is assembled into amyloid fibrils. Polymerisation into fibrils appears to require at least partial unfolding of native proteins and does not appear to be restricted to proteins whose native fold contains β-sheets. Indeed, formation of fibrils from native sheets of proteins is frequently associated with a conversion from helical to sheet structure.

Even in the case of the SH3 domain, where the native fold is largely β structure, the structure of the fibrils suggests that this must be substantially rearranged relative to that of the native protein.

### Example 2

### Example 2 (i)

Muscle acylphosphatase was purified as previously reported (A. Modesti et al. (1995) Protein Express Purif. 6, 799) and incubated at a concentration of 0.375 mg/ml (34 μM) in 25% v/v trifluoroethanol (TFE), acetate buffer, pH 5.5 at 25° C. under constant stirring. Aliquots were withdrawn at regular time intervals for electron microscopy and spectroscopic analysis. Circular dichroism spectra were acquired directly by means of a Jasco J-720 spectropolarimeter and cuvettes of 1 mm path length. Electron micrographs were acquired by a JEM 1010 transmission electron microscope at 80 kV excitation voltage. A 3 μL sample of protein solution was placed and dried for five minutes on a Formvar and carbon-coated grid. The sample was then stained with 3 μL 1% phosphotungstic acid solution and observed at magnifications of 25-100k.

### Example 2 (ii)

Infrared spectra were acquired using BaF₂ windows of 50 μm path length.

### Example 2 (iii)

Thioflavin T and Congo Red assays were performed according to Le Vine III (H. Le Vine III (1995) Amyloid: Int. J. Exp. Clin. Invest. 2,1.) and Klunk (W. E. Klunk et al. (1989) J. Histochem. Cytochem. 37, 1293), respectively. For Congo Red birefringence experiments aliquots of protein were air dried onto glass slides. The resulting films were stained with a saturated solution of Congo red and sodium chloride, corrected to pH 10.0 with 1% sodium hydroxide. The stained slides were examined by an optical microscope between crossed polarizers.

There is increasing evidence that amyloids develop not directly from the native and functional conformation of the protein, but from an amyloidogenic precursor bearing scant resemblance with the conformation of the native protein and identifiable in a denatured conformation containing a certain level of residual structure. This conformation is often referred to as amyloidogenic intermediate.

Muscle acylphosphatase is a protein that adopts, under physiological conditions, a well-defined fold, the stability of which is close to the average value for proteins of this size. Studies performed using trifluoroethanol (TFE) have revealed that muscle acylphosphatase is denatured at concentrations of TFE higher than 20-22% v/v. The denaturation of muscle acylphosphatase by TFE allows the maintenance of native α-helical structure of the protein and is accompanied by a virtual disruption of the hydrophobic core and by the concomitant formation of non-native α-helical structure. Further addition of TFE causes the accumulation of extra α-helical structure and the destabilisation of putative hydrophobic interactions that might be present under the lower alcohol concentrations. Therefore, an aqueous solution containing 25% v/v TFE, the lowest alcohol concentration at which the native protein is virtually absent, was chosen for fibril formation.

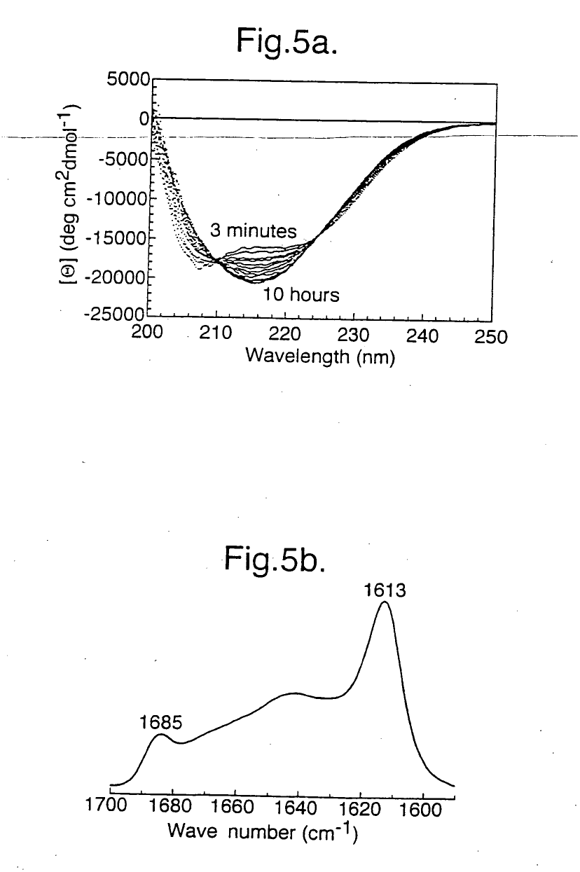

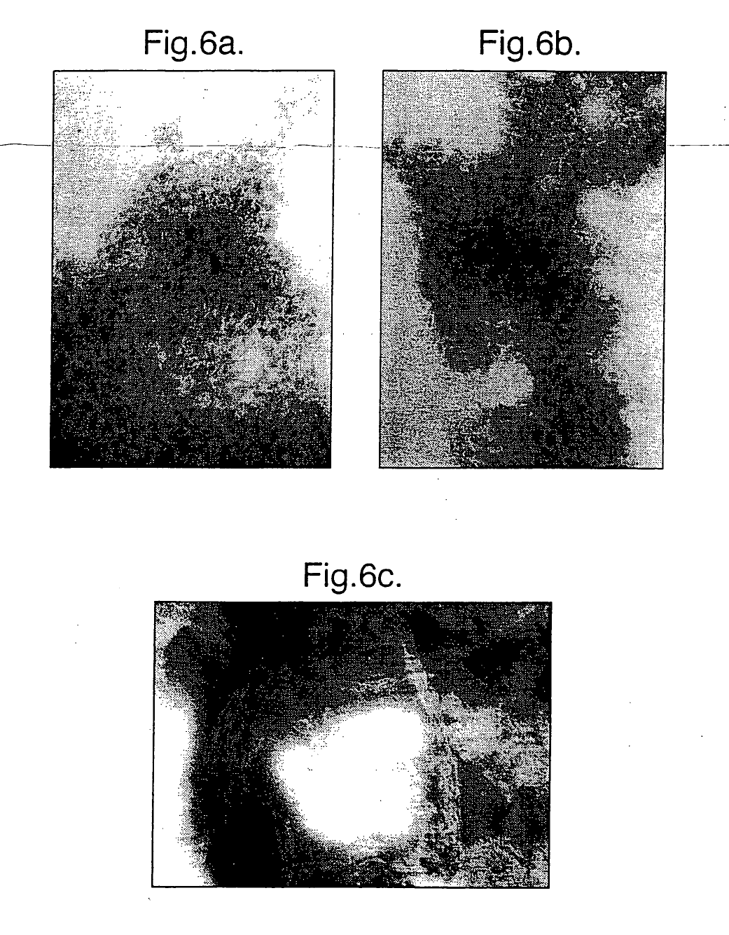

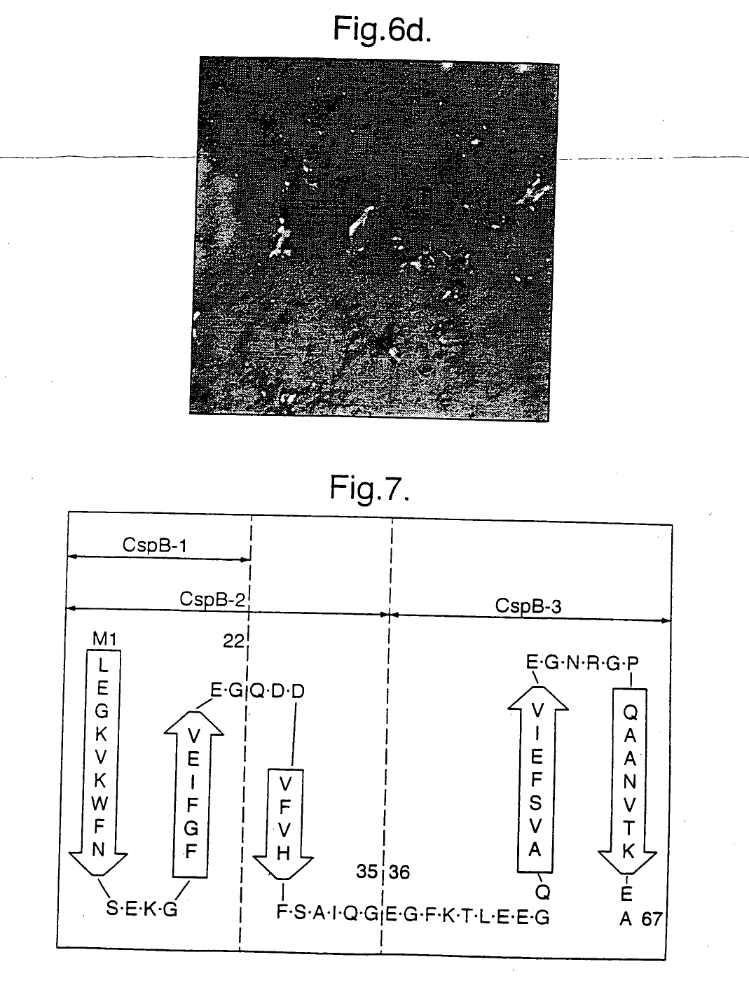

The sequence of events after mixing was probed by a variety of techniques including far-UV circular dichroism (CD), tryptophane intrinsic fluorescence, Congo Red and Thioflavin T binding, electron microscopy and Congo Red birefringence. Following the rapid denaturation of the protein, occurring on a time-scale of seconds, far-UV CD analysis revealed the presence of a slow transition, completed within 2-3 hours, from a conformation rich in α-helical structure to another containing a considerable content of β-sheet structure (FIG. 5A). Far-UV CD spectra were acquired at regular time intervals over this period. The first CD spectrum is typical of a conformation rich in α-helical structure with two negative peaks centred at 208 and 222 nm. This spectrum changes gradually to a β-sheet spectrum with a single negative peak around 216 nm (FIG. 5A) The presence of two isodichronic points at 210 and 225 nm suggests that such α/β transition consists of a two-state process. That such β-sheet structure derives from the intermolecular hydrogen bonding established within a protein aggregate is suggested by the two bands at 1685 and 1613 cm⁻¹ in the amide region of the infra-red spectrum (FIG. 5A) and by the electron micrographs revealing the presence of protein aggregates of granular aspect from samples recovered at this stage of the aggregation process (FIG. 6A).

After a period of ca. 32 hours the electron micrographs revealed the presence of short filaments, indicating that a fibrillar protein aggregate had grown to a significant extent. After two weeks the fibrillar material was more evident. The fibrils revealed by the electron micrographs were long, unbranched and 8.5 nm in width, whereas very short filaments or other protein aggregates of granular aspect were no longer present. A series of optical tests were performed to investigate further the amyloid nature of this fibrillar material. A three fold increase of the 482 nm fluorescence (excitation 440 nm) of the dye Thioflavin T was observed as a consequence of the addition of the protein aggregate, a result expected for amyloids. In addition, the protein aggregate produced a red-shift of the maximum light absorption of the dye Congo Red. The subtraction of the absorption spectra of the aggregate alone and Congo red dye alone from the spectrum containing both the aggregate and the Congo Red dye produces a spectrum, with a maximum intensity at 540 nm. These two findings are also indicative of the presence of amyloid fibrils. Finally, the addition of Congo Red to a sample with muscle acylphosphatase-derived fibrils produced the characteristic green birefringence under cross-polarised light (FIG. 6D). The development of green birefringence is highly diagnostic for the presence of amyloid fibrils. In summary, the muscle-acylphosphatase responded positively to all diagnostic tests for the presence of amyloid fibrils.

Recently, amphipathic compounds such as phospholipids have been suggested to facilitate the elongation of the fibrils. The formation of amyloid fibrils by fluoroalcohols like TFE supports this suggestion that such amphipathic compounds normally present in biologic systems might act as a medium for the growth of amyloid fibrils in vivo.

Concentrations of TFE lower than 20% or higher than 35% did not lead to fibril formation. This may be because the fibrillogenesis process is hindered by the presence of the native conformation of the protein at low TFE concentrations or by the presence of denatured states too rich in α-helical structure at high concentrations of TFE. These may reduce the concentration of the amyloidogenic precursor acting therefore as kinetic traps for the process of fibril formation. Very high protein concentrations may also constitute an obstacle to the process of the fibrillogenesis process. When incubated at concentrations higher than 3 mg/ml muscle acylphosphatase led to the rapid and irreversible formation of a gel-like precipitate that electron microscopy revealed to be an amorphous protein aggregate. Amyloidogenesis, like crystallogenesis, is a process in which the protein molecules self-assemble to form ordered structures. High protein concentrations may favour the concentrations of molecules and accelerate any aggregation process. Under such conditions, however, there may not be sufficient time for formation of ordered and repetitive conformations.

### Example 3

Peptide Synthesis

Peptides were assembled on an Applied Biosystems (Foster City, Calif.) 430A automated peptide synthesizer using the base-labile 9-fluorenylmethoxycarbonyl (Fmoc) group for the protection of the α-amino function.

Side-chain functionalities were protected by the t-Bu (Asp, Ser, Thr, Tyr), trityl (Asn, Gln), or the Pmc (Arg) group. Synthesis and purification were carried out by known methods. The identity and purity of the peptides were confirmed by ESI-MS. The masses measured for CspB-1, CspB-2, and CspB-3 were 2,531.1, 3,976.6 and 3,449.5 g/mol, respectively (masses predicted from the sequence: 2,531.9, 3,976.6, 3,448.7).

Sample Preparation

All three peptides were found to have optimal solubility if first dissolved in 50t acetonitrile pH 4.0 (adjusted with formic acid, unbuffered), and subsequently diluted to the desired peptide and acetonitrile concentrations.

Optical Spectroscopy

CD spectra were recorded on a Jasco J720 spectropolarimeter using quartz cuvettes of 1 mm pathlength, at 1 nm intervals from 195 to 250 nm. Routinely, CD samples were examined 30 min after dilution from the peptide stock solution containing 50% acetonitrile. Kinetic experiments revealed that, after this time, given the relatively low-concentration (0.4 mg/mL) of the CD samples, no time dependent effects could be observed on the timescale of minutes.

Calculation of the β-sheet content was carried out using the ellipticity at 215 nm normalized to a per residue basis (units: deg cm²/dmol). The highest value observed in this study (−9,260 deg cm²/dmol) was taken to correspond to 100% β structure, which agrees well with the value proposed by other workers (−9,210 deg cm²/dmol at 216 nm).

For the congo red binding assay of fibril formation, absorption spectra of a 10 μM solution of the dye in the assay buffer (5 mM phosphate pH 7.41 0.15 mM NaCl) before and after addition of the peptide solution were recorded on a Perkin Elmer (Foster City, Calif.) Lambda 16 spectrometer in the range of 400 to 700 nm. Typically, 10 μL of the peptide sample were used in a total volume of 1.0 mL.

NMR spectroscopy

All NMR spectra were acquired at ¹H frequencies of 500 or 600 MHz on homebuilt NMR spectrometers at the Oxford Centre for Molecular Sciences. One dimensional (1D) spectra typically contain 8K complex data points. Two dimensional (2D) experiments were acquired with 2K complex data points in the t₂ dimension, and in phase-sensitive mode using time proportional phase incrementation (TPPI) for quadrature detection in t₁. Diffusion constants were determined using pulse field gradient experiments with 8K complex points. Spectral widths of 8,000 Hz were used for all experiments. For the resonance assignments, DQF-COSY, TOSCY, ROESY, and NOESY spectra were recorded, involving between 512 and 800 t₁ increments with 32 to 128 scans each. The water signal was suppressed either by using presaturation during the 1.2 s relaxation delay or by using a gradient double echo. The mixing times for the TOCSY experiments varied between 23 and 60 ms, and for the NOESY and ROESY experiments between 100 and 260 ms. Data were processed using Felix 2.3 (BIOSYM) on Sun workstations. Typically, the data were zero filled once, and processed with a double exponential window function for the 1D, and a sinebell squared function, shifted over 90° for each dimension of the 2D spectra. All spectra were referenced to an internal standard of dioxan at 3,743 ppm.

For the determination of the ³J_{NHα coupling constants, high resolution DQF-COSY spectra were recorded with} 4K complex points zero filled to 8K, and the window functions GM 3.75 and TM 16 4096 4096 applied. The cross peaks were then fitted to simulated antiphase cross sections along the F2 dimension using a procedure implemented in the Felix 2.3 program.

A series of 1D spectra was used to estimate the percentage of peptide visible by solution NMR. These 1D spectra were all acquired with a gain of 7,000 and 256 scans on the same 500 MHz NMR spectrometer, in consecutive experiments. The concentration of a tryptophan solution was calculated from its absorbance at 280 nm. The integration of the indole resonances in these ID spectra from the tryptophan and peptide solutions was used to estimate the concentration of the peptide solutions.

Electron Microscopy

Fibril formation and morphology were examined by transmission electron microscopy (EM). Peptide samples were dried onto formvar-and carbon-coated grids and negatively stained with 1% phosphotungstic acid (PTA). Grids were examined in a JEOL JEM-1010 electron microscope at 80 kV excitation voltage.

Light Microscopy

Confirmation of the presence of amyloid fibrils in peptide samples was obtained by drying congo red stained fibrils onto glass slides and examining the preparations through a binocular microscope using crossed polarizers. Yellow green birefringence indicates the presence of cross p structure.

X-ray Fiber Diffraction

Droplets of 10 μL peptide solution were suspended between the ends of two capillaries sealed with wax. While the normal procedure involves evaporation of the solvent over a timescale of ˜1 day, the presence of acetonitrile in the samples allowed the droplet to evaporate in approximately 1 h. Diffraction patterns of the remaining solid in the form of two thin needles attached to the ends of the capillaries were collected using a Cu K_{α}rotating anode equipped with a 180 mm image plate (MAR Research, Hamburg, Germany). The diffraction pattern was analyzed using the Mar-View software (MAR Research).

Results

CspB has been shown by X-ray crystallography as well as by NMR to have a simple all β-sheet topology homologous to the S1 domain. Like S1 and the homologous Escherichia coli cold shock protein, CspA, the Bacillus protein has been shown to bind single-stranded RNA, and it is thought to act as an RNA chaperone in that it stops mRNA from forming unwanted secondary structure at low temperatures. CspB is remarkable for its ability to fold very rapidly, and for its relatively low contact order (i.e. a high proportion of contacts between residues close to each other in the linear sequence). As all the β-sheet forming interactions occur between strands that are neighbouring in the sequence, secondary structure formation might be possible both during the synthesis on the ribosome and also within the short peptide fragments investigated here.

Although this protein is not related to any of the at least 18 known eukaryotic constituents of pathological amyloid fibrils all three CspB peptides precipitate as fibrils with characteristics closely similar to mammalian amyloid from a variety of conditions where highly unstructured monomers are the prevailing species in solutions. It is possible that the initial secondary structure content of the monomeric polypeptide is not a major determinant of amyloid formation. The most important requirement may be the lack of ordered tertiary structure under conditions where interactions such as hydrogen bonds or hydrophobic contacts are still viable. This requirement may be met either by conditions that induce at least partial unfolding of the intact protein, or by dissecting a polypeptide chain into shorter peptides that are unable to form cooperative globular structure.

Based on the known structure of the B. subtilis cold shock protein CspB, consisting of five β-strands arranged in a small β sandwich, the peptides CspB-1 (residues 1-22), CspB-2 (1-35), and CspB-3 (36-67) were designed to correspond to the first two, the first three, and the last two β-strands of the CspB protein, respectively (see FIG. 7). While CspB-1 and CspB-2 represent a nascent protein growing from the N-terminus, CspB-2 and CspB-3 represent the two halves of the β sandwich and together cover the entire sequence length of the original protein.

Cold shock protein B is soluble in aqueous buffers at pH values ranging from 6.0 to 7.2 to a protein concentration of at least 1.3 mM (10 mg/mL), as demonstrated by the fact that the NMR structure was obtained under these conditions. In contrast, none of the three peptides are soluble under similar conditions to any significant extent; CspB-2, for example, dissolves only to ˜0.2 mg/mL. To solubilize the peptides, acetonitrile was used as a cosolvent at pH 4.0 (formic acid, unbuffered). All three peptides are soluble to 10 mg/mL or higher under these conditions, although their solubility decreases drastically if the acetonitrile concentration is changed to values significantly higher or lower than 50%. For example, attempts to prepare NMR samples by diluting stock solutions containing 20 mg/mL peptide in 50% acetonitrile with four volumes of either water or acetonitrile resulted in rapid precipitation of the peptides. A standardized set of combinations of peptide concentration and solvent composition was used, involving peptide concentrations of 0.4, 2 and 10 mg/mL, and acetonitrile concentrations of 10, 50 and 90%.

NMR studies of the three peptides were carried out under the conditions where they are most soluble (10 mg/mL peptide in 50% acetonitrile pH 4.0). CD studies were carried out at acetonitrile concentrations ranging from 5 to 95%. Due to the lower peptide concentrations needed for CD spectroscopy (0.4 mg/mL) spectra could still be obtained under conditions of relatively low solubility (i.e. in higher and lower concentrations of acetonitrile). The insoluble material produced by solvent shifts was analysed by a variety of techniques including specific tests for the presence of amyloid fibrils.

CD Measurements

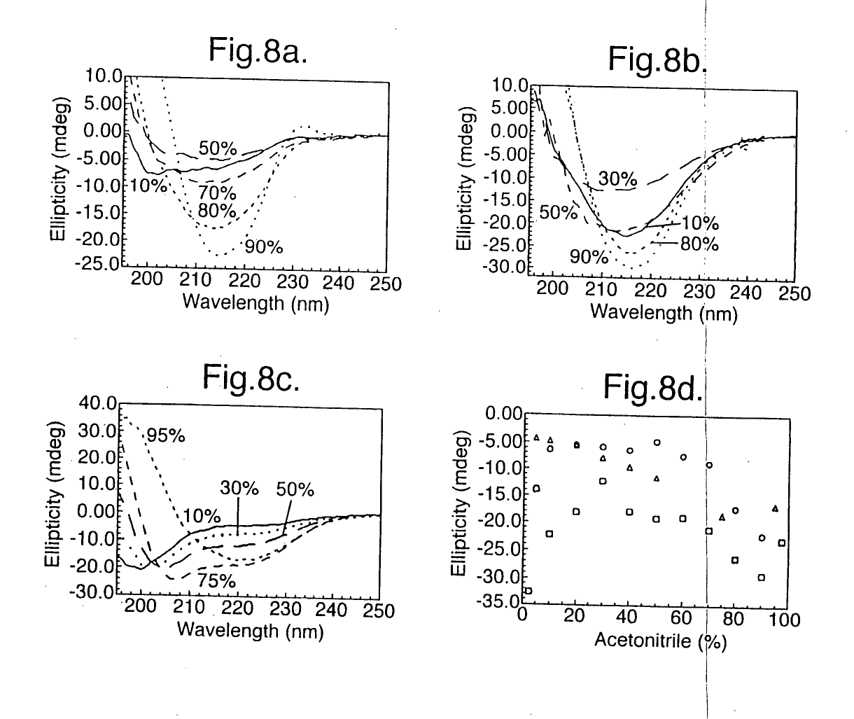

The three peptides were found to differ substantially in their structural properties, as monitored by CD spectroscopy, particularly under the conditions where they are relatively insoluble and from which fibril formation can be initiated. In the soluble state (50% acetonitrile), CspB-1 appears to be largely unstructured at 0.4 mg/mL (see FIGS. 8A and 8D) although it forms β-sheet structure at very high peptide concentrations, as indicated by additional CD measurements using a 0.1 mm pathlength cell. The ellipticity per residue increases from ˜1,730 to ˜6,480 deg cm²/dmol as the acetonitrile concentration is increased to 90%, suggesting ˜70% of structure at the highest acetonitrile concentration.

CspB-2 (FIGS. 8B and 8D) adopts a largely β-sheet conformation at very high and at very low acetonitrile concentrations (100% β-sheet at 2.5% acetonitrile, and 71.5% β-sheet at 97.5% acetonitrile). It is less structured at intermediate solvent conditions (mean residue ellipticities around −3,410 deg cm²/dmol, indicating ˜20% β-sheet content in the range from 15 to 70% acetonitrile). CspB-3 (FIGS. 8C and 8D) displays particularly interesting behaviour as it can be in predominantly unstructured, partly helical, or largely β-sheet conformations depending on the acetonitrile concentration. The CD data show that the helix content increases gradually as the acetonitrile concentration is increased from 5 to 75%, but the peptide converts to predominant β-sheet structure at acetonitrile concentrations between 75 and 95%. At the latter concentration, the ellipticity per residue observed at 215 nM for this peptide is −3,830 deg cm¹/dmol, corresponding to 41% β-sheet.

At low concentrations of acetonitrile (10%), CspB-2 adopts a β-sheet conformation, while the other two peptides are predominantly unstructured. At high acetonitrile concentrations (90%), however, all peptides show some extent of β-sheet formation, which overlaps with random coil properties for the two N-terminal peptides, and with α-helical structure for the C-terminal peptide (FIG. 8). CspB-3 changes sequentially from unstructured to helical to β-sheet conformation when gradually transferred from 5 to 95% acetonitrile. Generally, therefore, the β-sheet content increases when the conditions change toward lower solubility or higher acetonitrile concentration. This is indicative of intermolecular rather than intramolecular β-sheet formation. The data suggest that the monomers are mostly unstructured (or partially helical in the case of CspB-3), while the aggregates all contain 13 structure.

NMR Experiments

NMR diffusion measurements conducted at a peptide concentration of 10 mg/mL in 50% acetonitrile indicate a single diffusion constant for each peptide, corresponding to values of hydrodynamic radii similar to those predicted for unfolded monomers (see Table 1).

TABLE 1
Hydrodynamic radii^{a} of CspB peptides at 10 mg/mL
concentration in 50% acetonitrile, as determined by NMR
diffusion measurements

| | Hydrodynamic | Predicted Value | Predicted |
|---|---|---|---|
| Peptide | Stokes radius | unfolded | Value unfolded |
| (No. of residues) | (nm)^{b} | monomer (nm)^{c} | dimer (nm) |
| CspB-1(22) | 1.60^{a} | 1.29 | 1.80 |
| CspB-2(35) | 1.68 | 1.72 | 2.54 |
| CspB-3(32) | 1.66 | 1.63 | 2.43 |

^{a}Hydrodynamic radii were calculated using Stokes' law from diffusion measurements. The Stokes radius of CspB-1 was also determined in the supernatant of a sample containing 2 mg/mL peptide in 10% acetonitrile after fibril formation was completed. The Stokes radius of this species was 1.34 nm.
^{b}HPLC analysis confirms that the peptides in their soluble states are largely monomeric, but provides evidence for minor populations of small oligomers. In chromatographic profiles, monomers are the dominant species, while dimers and higher order oligomers are present only at lower concentrations (<30%).
^{c}Predictions are based on a fit of the hydrodynamic radii measured by pulse field gradient NMR for highly unfolded proteins and peptides, resulting in the empirical equation: R = 0.225N^{0.57} where R is the hydrodynamic radius in nanometers and N is the number of amino acid residues.

This suggests that the samples consist predominantly of monomers. Any small oligomers (whose signals merge with the monomer signal if conformational interconversion takes place on a time scale shorter than that resolvable by this method, i.e. less than ˜100 ms) can only exist as minor populations. Calibration of the spectral intensity, however, indicates that the spectral intensities are lower than expected for the concentration of peptide involved. This was examined quantitatively for CspB-1, where it was found that only ˜20% of the total peptide concentration present in the sample is detectable in the NMR spectra. The remainder must therefore be in large soluble aggregates whose overall tumbling times are too large to give resolvable NMR resources.

Detailed structural NMR studies of all three peptides were undertaken at 20 and at 35° C. in solutions containing 50% acetonitrile. For all peptides, complete assignment of the amide resonances was achieved by analysis of COSY and TOCSY spectra acquired at 35° C. None of the peptides showed any measurable long range NOEs or ROEs. This indicates the absence of any significant persistent structure. The structural characteristics of the peptides were therefore inferred by comparing their chemical shifts and coupling constants to those predicted from random coil models. Although random coil values of chemical shifts are well documented the values obtained for amide protons are notoriously dependent on solvent conditions.

Therefore, we used only the C_{α}proton shifts in this analysis. These show only minor deviations from typical random coil values (most between ±0.1, and all between ˜0.20 and +0.15 ppm).

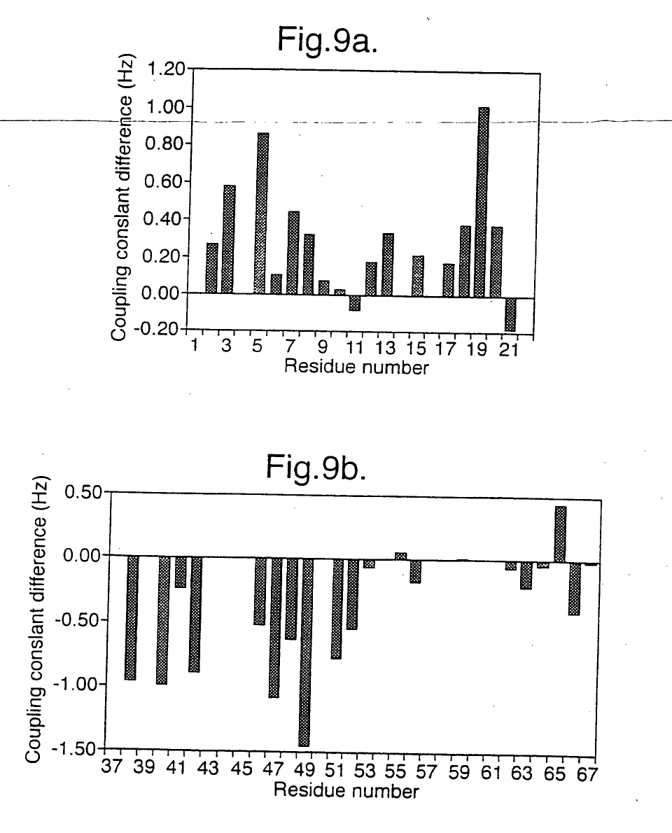

Most of the coupling constants measured for CspB-1 are slightly larger than the values predicted for a random coil (FIG. 9A). This indicates that CspB-1 has a slightly higher occupancy of the β-region of φΨ space than anticipated for a random coil. This is most pronounced in the regions of residues 5-9 and 17-20 (FIG. 9A). Both these groups of residues are within the regions of the β-strands in the native protein (2-10, 15-20). For CspB-3, the coupling constant measurements show smaller values than predicted for a random coil, suggesting that the small amount of helical structure observed in the CD spectra is likely to be localized between residues 38 and 53 (FIG. 9B). This result is also in accord with the slight propensity for helix formation in this region revealed by several of the secondary structure prediction methods used. Thus, the Gibrat, Levin, DPM, and SOPMA methods predict the conformation of the majority of the residues in positions 38 to 47 to be helical (data not shown). Additional helicity, which is predicted to occur near the carboxy terminus, was not observed by NNM.

Evidence for Amyloid Fibril Formation

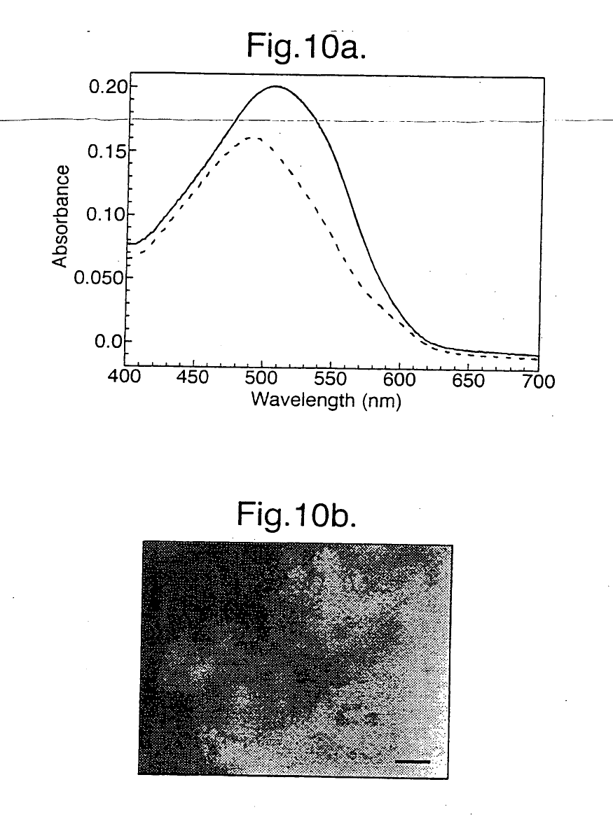

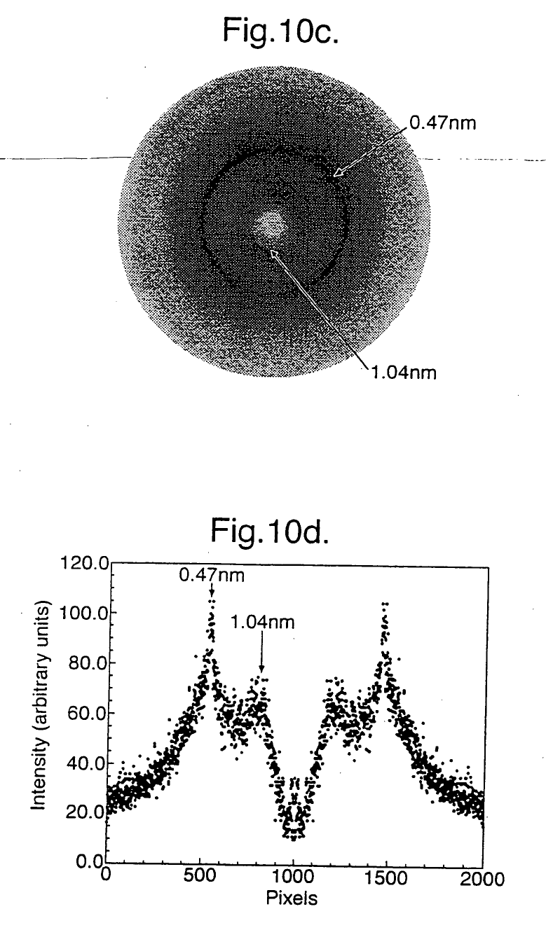

Analysis by three independent techniques of samples produced by dilution of concentrated solutions of all three peptides (10.0 mg/mL diluted to 2.0 mg/mL final concentration) from 50% to either 10 or 90% acetonitrile was carried out to screen for the presence of amyloid fibrils. CspB-1 was used for all further investigations into fibril formations. Spectroscopic binding assays using the diazo dye congo red (FIG. 10A) show the typical red shift in wavelength and increase in intensity characteristic of amyloid fibrils. In transmission electron microscopy (FIG. 10B), a dense network of straight and unbranched fibrils approximately 10 nm in diameter and up to 300 nm long was observed for CspB-1 diluted into 10% acetonitrile. Under other experimental conditions, and with the other peptides, fewer fibrils were observed, but similar morphologies were evident. Light microscopy of congo red stained precipitates using crossed polarizers (now shown) revealed the green bireffingence characteristic of amyloid fibrils.

Aggregated states of all three peptides produced in 10 and 90% acetonitrile were analysed using these techniques. While all types of experiment were strongly positive for the presence of amyloid for CspB-1 in 10% acetonitrile, more varied results were obtained under some of the other conditions. Nevertheless under all conditions positive evidence indicating formation of amyloid fibrils was obtained(see Table 2).

TABLE 2
Overview of results obtained a series of two
screening experiments to assess the formation of amyloid
fibrils under different conditions, using congo red
binding, EM, and detection of the green birefringence by
light microscopy (LM)^{a}

| Condition | Method | CspB-1 | CspB-2 | CspB-3 |
|---|---|---|---|---|
| 10% acetonitrile | Congo Red | ++, ++ | ++, 0 | ++, ++ |
| | EM | ++ | ++ | + |
| | LM | ++, ++ | + | ++, + |
| 90% acetonitrile | Congo Red | ++, 0 | 0, ++ | ++, + |
| | EM | 0 | ++ | 0 |
| | LM | ++, ++ | ++, 0 | 0, ++ |

^{a}The final peptide concentration was 1.6 mg/mL for the first congo red experiment carried out under each set of conditions and 2.0 mg/mL for all other experiments.
Results:
(++) strongly positive,
(+) weakly positive,
(0) no positive evidence.
In addition to the results shown here, fibril formation in 10% acetonitrile was observed in many more experiments carried out with CspB-1 in separate studies.

Further examination was carried out with the fibrils formed by CspB-1 in 10% acetonitrile. Intermolecular β-sheet structure, which is compatible with fibril formation, was also demonstrated by FTIR for peptide CspB-1. X-ray fiber diffraction also yielded results characteristic of amyloid fibrils. For the latter technique, a sample of CspB-1 in 50% acetonitrile was left to dry down after suspending between two capillaries. As a result of the higher volatility of acetonitrile, the solvent composition changes during the evaporation and is expected to be near 10% acetonitrile during the actual precipitation process. This resulted in a thin needle of precipitate, which showed X-ray diffraction patterns (FIG. 10C and 10D) with diffraction maxima at 0.47 and 1.04 nm, typical of amyloid fibrils.

Thus three peptides derived from a small bacterial protein.with an all β structure can form amyloid fibrils. The formation of these fibrils can occur from quite different starting situations by solvent shifts toward higher or lower concentrations of acetonitrile. Typically, the solutions contain populations of largely unstructured monomers, together with oligomers and soluble aggregates containing significant amounts of β-sheet structure. This suggests that amyloid formation does not depend on the presence of extensive preformed secondary structure elements within monomeric species in solution, although the aggregates and the amyloid fibrils themselves contain extensive β-sheet structure. More generally, β structure is common within a wide range of aggregates of different morphologies. The ability to form aggregates with such β structure is likely to be an important factor in the subsequent conversion to ordered amyloid fibrils.

### Example 4

WT-ADA2h Expression and Purification

The activation domain of wild type human carboxypeptidase A2 (WT ADA2h) was expressed and purified as previously reported. The recombinant protein was examined by MALDI-TOF-MS and found to have the molecular weight anticipated from the sequence.

Circular Dichroism

CD spectra of 20, 80, 160 and 200 μM protein samples in 50 mM sodium phosphate (pH 7.0) or 25 mM glycine (pH 3.0) were recorded using a JASCO-7 10 spectropolarimeter, at 278, 298 and 368° K in a 2.0 or 0.2 mm quartz cuvette. Measurements were averaged for 30 scans recorded at 50nm min⁻¹. Thermally induced unfolding of 20 μM protein samples was monitored in the temperature range of 278-368 K at a heating rate of 50° Ch^{31 1} by following their ellipticity at 222 nm or 214 nm.

Sedimentation Analysis

Sedimentation experiments were performed in a Beckman XLA analytical ultracentrifuge at 3000 g with 20 and 200 μM protein samples in a buffer solution at pH 3.0 containing 25 mM glycine. Samples were heated at a rate of 50° Ch^{31 1} from 5 to 95° C. and then left at 95° C. for 10 min before sedimentation experiments. A 200 μM protein sample in 50 mM sodium phosphate at pH 7.0 was used as negative control.

Thioflavine-T and Congo Red Binding Assays

Samples of WT-ADA2h (at concentrations ranging from 20 to 500 μM) were incubated for 30 min at 90° C. before the assay. A 2.5 mM Thioflavine-T stock solution was freshly prepared in 10 mM potassium phosphate, 150 mM NaCl, pH 7.0, and passed through a 0.2 μm filter before use. Typically 10 μl of sample was diluted in the reaction buffer (10 mM potassium phosphate, 150 mM NaCl, pH 7.0) containing. 65 μM Thioflavine-T (1 ml final volume). Samples were taken into and out of the pipette several times to facilitate fibril dispersion and to disrupt large aggregates. Data were collected in a Perkin-Elmer LS 50 B luminescence spectrometer using a 440 nm (slit width 5 nm) excitation wavelength and 482 nm (slit width 10 nm) emission wavelength. Fluorescence values were obtained after 3-5 min to ensure thermal equilibrium had been achieved. Samples were continuously stirred to prevent signal oscillation due to the presence of large fibrillar aggregates and signals were averaged for 60 s to increase the signal-to-noise ratio. Congo red binding assays were performed according to Klunk et al. Aliquots of 10 μl of sample were diluted in 10 μM potassium phosphate, 150 mM NaCl, pH 7.0 containing 5 μM Congo red (1 ml final volume). Congo red solutions were prepared just before use and passed through a 0.2 μm filter. Absorption spectra of samples in the reaction solution were collected together with negative controls (dye in the absence of protein and protein samples in the absence of dye) to subtract the signal associated with the absorption of the dye and the scattering contribution to the signal.

Proteinase Resistance

20 μM Samples of WT-ADA2h in 25 mM glycine (pH 3.0) were incubated for 4 h in 4 M urea, then diluted to give an urea concentration of 1 M, and dialysed before incubation with proteinase. Samples in the same buffer were also incubated for 10 min at 95° C. before proteolysis. These sample together with untreated samples of the same protein were digested in the presence of pepsine at two different WT-ADA2h:pepsine ratios (100:1 and 400:1) for 2 h at 20° C. or 15 min at 0° C. respectively. Digested samples were then analysed by Rβ-HPLC in a Vydac C4 column (214TP54,5 μm particle size, 300 Å pore, 1.0×25 cm) with a linear gradient from 10 to 52% of acetonitrile. Detection was carried out at 214 nm in a Waters 994 model.

Fourier-transform Infrared Spectroscopy

Infrared spectra were recorded in a Bio-Rad FTS 175C FT-IR spectrometer equipped with a liquid N₂-cooled MCT detector, and purged with a continuous flow of N₂ gas. 500μM WT-ADA2h samples were prepared in ²H₂O, glycine 25 mM, p²H 3.0 (the electrode reading was corrected for isotope effects), and spectra were collected at 25° C. before and after incubating the sample at 90° C. for 30 min. Protein solutions were placed between a pair of CaF² windows separated by a 12μm Mylar spacer. For each sample 256 interferograms were collected at a spectral resolution of 2 cm⁻¹. Spectra were collected under identical conditions for the buffer solution in the absence of protein and subtracted from the spectra of the protein samples. Second derivatives of the Amide 1 band spectra were produced to determine the wavenumbers of the different spectral components.

Electron Microscopy

Samples were applied to Formvar-coated nickel grids (400 mesh), negatively stained with 2% uranyl acetate (w/v), and viewed in a JEOL TEM 1010 transmission electron microscope, operating at 80 kV.

X-ray Diffraction

Fibril suspensions were washed with Microcon-100 ultrafiltration tubes (Amicon), to eliminate salts and buffers that could interfere with the X-ray measurements. Samples were prepared by air-drying salt depleted ADA2h-WT fibril preparations between two wax-filled capillary ends. The capillaries were separated slowly while drying, to favour fibril orientation along the stretching axis. A small stalk of fibrils protruding from one end of the capillaries was obtained. The sample was aligned in a X-ray beam, and diffraction images were collected in Cu Ka rotating anode equipped with a 180 or 300 MAR-Research image plate (MAR Research, Hamburg, Germany) during 20-30 min. Images were analysed by using IPDISP and MarView Software.

Results

This example relates to studies of an 81-residue protein, the activation domain of wild type human carboxypeptidase A2, WT-ADA2h. This domain has two α-helices packed against a four-stranded β-sheet. It has been found to fold at neutral pH in a two state manner through a compact transition state, possessing some secondary structure and a rudimentary hydrophobic core.

Thermal denaturation at pH 7.0 of WT-ADA2h has been shown to be reversible and follows a two-state transition. At pH 3.0 the WT-ADA2h undergoes an unfolding, transition (in a concentration range 10 μM to 3 mM) that is not reversible. The CD spectra at 95° C., and after cooling to 25° C., show that the protein has converted to a conformation with extensive β-sheet structure. Analytical centrifugation analysis indicates that the sample is highly aggregated (40% of the sample showed a S_{W,20} of 35 S, thus indicating a Mᵣ higher than 10⁶ Da, corresponding to an aggregate that contains on average more than 100 protein molecules). The conversion of α-helical structure to β-sheet for the WT-ADA2h protein upon thermal denaturation is corroborated by FT-IR spectroscopy. Before heating, the amide I band shows two main components at 1622 and 1649 cm⁻¹ respectively, attributable to β-sheet and α-helical structure respectively, and consistent with the native state of the protein. After incubating the sample at 90° C. two new bands appear, centred at 1615 and 1685 cm^{31 1} respectively, replacing the original bands. This pattern is normally associated with aggregated species with sheet structures. The band at 1615 cm⁻¹ is indicative of β-sheet whereas the one at 1685 cm⁻¹ is associated with a splitting in the amide I band due to antiparallel inter strand interactions. The protein after heating also produces a clear shift in the Congo red absorbance spectrum from 486 to 500 nm, together with an increase in absorption, and exhibits thioflavine-T binding (Table 3). All these properties are consistent with the formation of amyloid deposits.

TABLE 3
Thioflavine-T binding of WT-ADA2h samples,
before and after thermal denaturation at pH 3.0. All the
samples were prepared in 25 mM glycine, pH 3.0 and
incubated for 30 minutes at the indicated temperatures.

| Sample Conditions | WT-ADA2h^{a} |
|---|---|
| 25° C. 500 μM | 3.1 |
| 90° C. 500 μM | 520.1 |
| 90° C. 200 μM | 288.7 (721.8)^{b} |
| 90° C. 100 μM | 111.5 (557.4) |
| 90° C. 50 μM | 64.3 (642.5) |

^{a}Results show the increase in fluorescence (arbitrary units) after subtracting the Thioflavine-T contribution alone (45.2 units).
^{b}Data in brackets represent the value normalised for the same concentration.
The behaviour of the protein was also examined following incubation in the presence of a range of urea concentrations (4 to 7M). At 7M urea the CD spectrum of the protein is indicative of a highly unfolded species (decrease in ellipticity at 222 nm).

When a sample of WT-ADA2h in 7 M urea is diluted rapidly to a concentration of 1 M urea, the protein refolds to its native state. Incubation of the WT-ADA2h protein in 4 M urea for 1 h, followed by dilution to 1 M urea and incubation for 1 h, however, shows a different CD spectrum indicative of extensive β-sheet structure.

The WT-ADA2h protein shows a reduced susceptibility to digestion pepsin in all the samples where aggregation has been detected (Table 4). Moreover, a clear correlation exists between the transition to β-structure revealed by CD and FT-IR and an increased resistance to proteolysis.

TABLE 4
Resistance to proteolysis of the WT-AD2h
aggregates. The data are given as the percentage of the
intensity in the chromatographic peak corresponding to
the native protein remaining after treatment.

| | Temperature | Urea | |
|---|---|---|---|
| | denatured | denatured | Untreated |
| Severe | 5.0% | 0.7% | 0% |
| proteolysis^{a} | | | |
| Mild | 39.7% | 7.4% | 1.5% |
| proteolysis^{b} | | | |

^{a}Severe conditions: Ratio WT-ADA2h:pepsin 100:1 (W:W), 2 h digestion, 20° C.
^{b}Mild conditions: Ratio WT-ADA2h:pepsin 400:1 (W:W), 15 min digestion, 0° C.

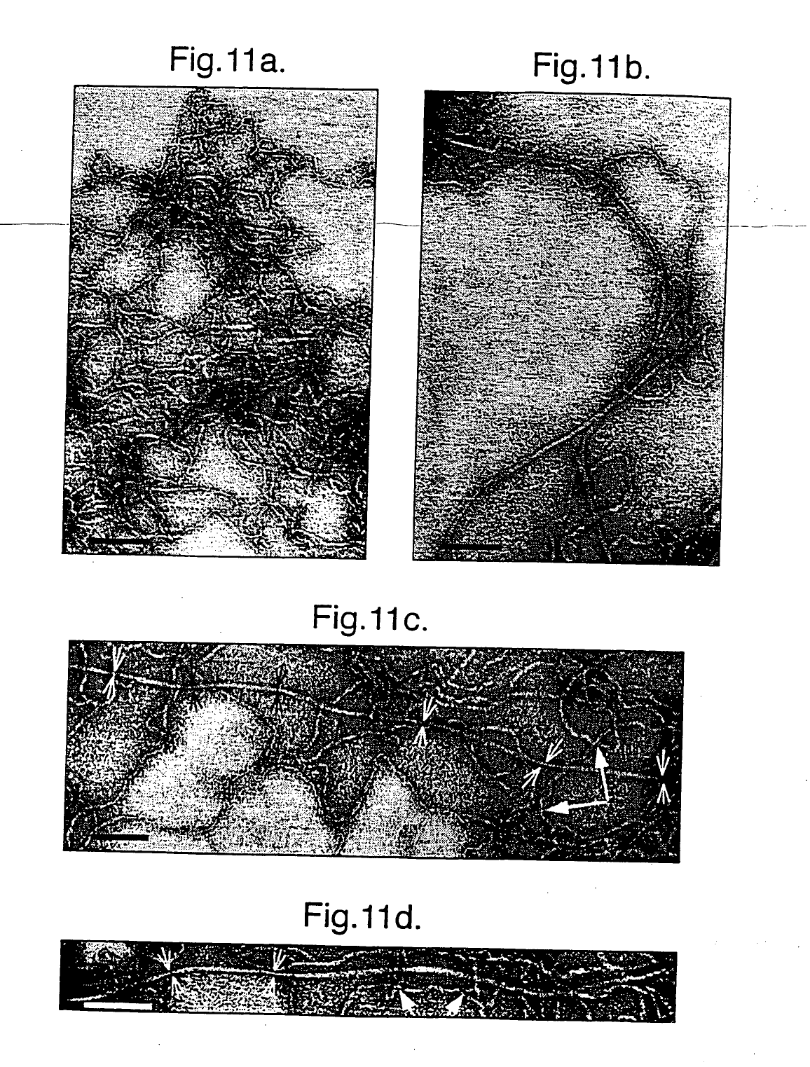

Analysis of the aggregated WT protein by electron microscopy shows clear evidence for fibrils that are long, unbranched, narrow (diameter 30-100 Å) and apparently quite flexible. These typically form tight networks of intertwined structures (FIG. 11a). Samples prepared after longer periods of incubation of 90° C. feature longer and more regular fibrils (FIG. 11b to d) that show more clearly a ribbon-like pattern twisting at irregular intervals (FIGS. 11b to d, see arrows). The structural differences arising from longer periods of incubation at high temperatures may be explained as a further slow reorganisation of the protofilaments which makeup the fibrils. Such structural evolution of the fibrils with time has been observed previously in amyloid fibrils produced from several other proteins. Fibrils are observable in aggregates formed from solutions containing protein concentrations as low as 20 μM. Fibrils with characteristics similar to those shown in FIG. 11a were also observed in samples of WT-ADA2h subjected to chemical denaturation, in agreement with the appearance of resistance to proteolysis.

Fibrils from the different thermally denatured preparations of WT-ADA2h were also characterised by fiber X-ray diffraction. These show a clear cross-β X-ray diffractions pattern, characteristic of amyloid fibrils with reflections at 4.7 Å (corresponding to the inter-strand distance in the direction of the fibril axis) and 9.3 Å (corresponding to the distance between β-sheet in the direction perpendicular to the fibril axis). Some anisotropy can be observed in the sharp 9.3 Å reflection. Another faint reflection can be observed at 3.1 Å, with the anisotropy and sharpness of the 9.3 Å one, probably arising from a harmonic. A fourth weak reflection is observed at 3.8 Å with no apparent anisotropy. A reflection of this type has been observed previously in studies of amyloid fibrils from a variety of sources.

WT-ADA2h, therefore, constitutes a further example of a protein not associated with any known disease that is able to aggregate in the form of amyloid fibrils when its native fold is destabilised.

I claims:

1. An amyloid fibril substantially free of other protein.

2. A fibril according to claim 1 which is a naturally occurring amyloid fibril.

3. A fibril according to claim 2 which comprises the Aβ peptide associated with Alzheimer's disease, the prion protein associated with the transmissible spongiform encephalopathies, the islet-associated polypeptide associated with type II diabetes, transthyretin and fragments thereof associated with senile systemic amyloidosis, transthyretin variants and fragments thereof associated with familial amyloidotic polyneuropathy or other variant, truncated, or misprocessed proteins associated with the systemic amyloidoses.

4. A fibril according to claim 1 which comprises a pharmaceutically active compound.

5. A fibril according to claim 1 which comprises a metal.

6. A fibril according to claim 1 which comprises a metal selected from copper, silver or gold.

7. A fibril according to claim 1 which comprises one or more functional groups capable of binding one or more reactants.

8. A fibril according to claim 1 wherein the diameter of the fibril is from 1 to 20 nm.

9. A fibril according to claim 1 wherein the diameter of the fibril is from 5 to 15 nm.

10. A fibril according to claim 1 wherein the diameter of the fibril is from 7 to 12 nm.

11. A non-naturally occurring amyloid fibril comprising a protein.

12. A fibril according to claim 11 wherein the protein is a non-naturally occurring protein.

13. A fibril according to claim 11 wherein the protein is selected from the group consisting of an SH3 domain (PI3-SH3) of a p85α subunit of bovine phosphatidylinositol 3-kinase, human muscle acylphosphatase, bovine insulin, a protein corresponding to the first two (CspB-1), the first three (CspB-2) or the last two (CspB-3) β strands of CspB, the wild type human carboxypeptidase A2 (WT-ADA2h) and derivatives or amino acid variants thereof.

14. A non-naturally occurring amyloid fibril comprising an SH3 domain (PI3-SH3) of a p85α subunit of bovine phosphatidylinositol 3-kinase and at least one protein selected from the proteins as described in claim 13.

15. A fibril according to claim 11 which further comprises a pharmaceutically active compound.

16. A fibril according to claim 11 which further comprises a metal.

17. A fibril according to claim 11 which further comprises a metal selected from copper, silver or gold.

18. A fibril according to claim 11 which further comprises one or more functional groups capable of binding one or more reactants.

19. A fibril according to claim 11 wherein the diameter of the fibril is from 1 to 20 nm.

20. A fibril according to claim 11 wherein the diameter of the fibril is from 5 to 15 nm.

21. A fibril according to claim 11 wherein the diameter of the fibril is from 7 to 12 nm.

22. A process for preparing an amyloid fibril, which process comprises:
preparing a solution comprising a protein, said solution being in a state so that nucleation and fibril growth will occur, and
allowing nucleation and fibril growth to take place.

23. A process according to claim 22 wherein the solution further comprises an alcohol.

24. A process according to claim 22 wherein the solution further comprises alcohol selected from methanol, ethanol, propanol, butanol, trifluoroethanol and hexafluoroisopropanol.

25. A process according to claim 22 wherein the solution further comprises acetonitrile.

26. A process according to claim 22 wherein the solution further comprises urea.

27. A process according to claim 22 wherein the concentration of protein in the solution is from 0.1 mM to 10 mM.

28. A process according to claim 22 wherein the temperature of the solution is from 0° C. to 100° C.

29. A process according to claim 22 wherein the solution is acidic.

30. A process according to claim 22 wherein the pH of the solution is from 0.5 to 6.5.

31. A process according to claim 22 wherein the solution is seeded with previously formed particles of protein.

32. Use of a fibril according to claim 1 as a plastic or in electronics or catalysis.

33. Use of a fibril according to claim 11 as a plastic or in electronics or catalysis.

34. A method of treating a human or animal, which method comprises administering thereto a non-toxic and effective amount of an amyloid fibril.

35. A method according to claim 34 wherein the human or animal is suffering from or susceptible to diabetes, blood clotting disorders, cancer or heart disease immediately prior to the administering.

36. A method according to claim 34 wherein the amyloid fibril is substantially free of other protein.

37. A method according to claim 36 wherein the amyloid fibril comprises a protein.

38. A process according to claim 22 wherein a non-naturally occurring amyloid fibril is prepared by said process.

39. A process according to claim 38 wherein the pH of the solution is from 0.5 to 6.5, the temperature of the solution is from 0° C. to 1000° C., and wherein the solution optionally also comprises an additive selected from the group consisting of an alcohol at 5 to 40% v/v, an aliphatic nitrile at 5 to 95% v/v and urea at 4 to 7 M.

40. A process according to claim 39 wherein said solutionis in a state so that the protein is at least partially denatured but self-association of the protein can still occur, and
wherein the process is selected from the group consisting of:
(a) a process wherein the protein is an SH3 domain of a p85α subunit of PI3 kinase;
(b) a process wherein the protein is muscle acylphosphatase, the solution has 5-40% v/v trifluoroethanol, the solution has acetate buffer, and the allowing step extends for at least 32 hours;
(c) a process wherein the protein is CspB-1, CspB-2 and/or CspB-3, and the solution has 10-90% acetonitrile;
(d) a process wherein the protein is carboxypeptidase, the solution contains glycine, and the allowing step extends for at least 30 minutes; and
(e) a process wherein the protein is carboxypeptidase A2, and the solution has 4 M to 7 M urea.
